(19) United States
(12) Patent Application Publication
(10) Pub. No.: US 2014/0171455 A1
(43) Pub. Date: Jun. 19, 2014

(54) Reduction of Microglia-Mediated Neurotoxicity by Kv1.3 Inhibition

(71) Applicants: Heike Wulff, Davis, CA (US); Lee-Way Jin, Davis, CA (US); Izumi Meezawa, Davis, CA (US)

(72) Inventors: Heike Wulff, Davis, CA (US); Lee-Way Jin, Davis, CA (US); Izumi Meezawa, Davis, CA (US)

(73) Assignee: The Regents of the University of California, Oakland, CA (US)

(21) Appl. No.: 14/124,226

(22) PCT Filed: Jun. 8, 2012
(86) PCT No.: PCT/US2012/041699

§ 371 Date: Mar. 7, 2014

Related U.S. Application Data
(60) Provisional application No. 61/495,350, filed on Jun. 9, 2011.

(51) Int. Cl. A61K 31/37; A61K 31/4709; A61K 31/517; A61K 31/353; A61K 31/47; A61K 31/472

(52) U.S. Cl. CPC A61K 31/37; A61K 31/47; A61K 31/472; A61K 31/517; A61K 31/353; A61K 31/4709
USPC 514/266.3; 514/455; 549/282; 514/312; 546/153; 514/307; 546/139; 544/287; 549/401; 514/456

(57) ABSTRACT

Methods for deterring microglia-mediated neurotoxicity in a human or non-human animal subjects comprising the step of inhibiting or blocking the intermediate-conductance calcium-activated potassium channel Kv1.3 in microglia, such as in subjects how suffer from neurodegenerative diseases (e.g., Alzheimer's Disease) or ischemic/anoxic/hypoxic conditions. The inhibition or blocking of the KCa1.3 channels may be accomplished by administering a substance that inhibits Kv1.3 in microglia. Examples of Kv1.3 inhibiting substances include certain 5-phenoxyalkoxypsoralens, such as (4-Phenoxybutoxy)psoralen (PAP-1) as well as certain 4-phenoxybutoxy-substituted heterocyclic compounds.

### RELATED APPLICATIONS

This patent application is the national stage filing under 35 U.S.C 371 of PCT International Patent Application No. PCT/US2012/41699 entitled Reduction of Microglia-Mediated Neurotoxicity by Kv1.3 Inhibition, filed Jun. 8, 2012, which claims the benefit of and right of priority to claims priority to U.S. Provisional Patent Application No. 61/495,350, filed Jun. 9, 2011, the entire disclosures of which are expressly incorporated herein by reference. Additionally, this application is a continuation-in-part of copending U.S. patent application Ser. No. 12/939,912 entitled 4-Phenoxybutoxy-Substituted Heterocycles and Their Use as Inhibitors of the Kv1.3 Lymphocyte Potassium Channel filed Nov. 4, 2010, now abandoned, which claims priority to U.S. Provisional Patent Application No. 61/258,134 filed Nov. 4, 2009 and is a continuation in part of U.S. patent application Ser. No. 12/498,334 entitled 5-Phenoxyalkoxypsoralens and Methods for Selective Inhibition of the Voltage Gated Kv1.3 Potassium Channel filed Jul. 6, 2009 and now issued as U.S. Pat. No. 8,067,460 which is a continuation of U.S. patent application Ser. No. 10/958,997 entitled 5-Phenoxyalkoxypsoralens And Methods For Selective Inhibition Of The Voltage Gated Kv1.3 Potassium Channel filed Oct. 4, 2004 and now issued as U.S. Pat. No. 7,557,138, the entire disclosure of each such application and patent being expressly incorporated herein by reference.

### STATEMENT REGARDING GOVERNMENT SUPPORT

This invention was made with Government support under Grant No. R21 AG038910 awarded by the National Institutes of Health. The Government has certain rights in the invention.

### FIELD OF THE INVENTION

This invention relates generally to the fields of chemistry, pharmacology and medicine and more particularly to the treatment of neurodegenerative diseases, deterring or reducing neuronal damage following ischemic/hypoxic/anoxic events and treatment of other conditions wherein microglia-mediated neurotoxicity occurs.

### BACKGROUND OF THE INVENTION

Pursuant to 37 CFR 1.71(e), this patent document contains material, which is subject to copyright protection. The copyright owner does not object to facsimile reproduction of the entire patent document, as it appears in the Patent and Trademark Office patent file or records, but otherwise reserves all copyright rights whatsoever.

Microglia are non-neural, interstitial cells of mesodermal origin that form part of the supporting structure of the central nervous system in humans and other mammals. Microglia are tissue resident macrophages of the brain. Microglia come in various forms and may have slender branched processes. They are migratory and, when activated (usually by some instigating stimulus), can act as phagocytes, which engulf and remove nervous tissue waste products.

In various neurodegenerative diseases, damage to nerve cells is believed to occur, at least in part, due to activation of microglia by some instigating stimulus (an “activator”). For example, in Alzheimer's disease (AD), amyloid plaques accumulate between nerve cells (neurons) in the brain. Amyloid is a term, which broadly refers to protein fragments that the body produces normally. Beta amyloid (Aβ) is a protein fragment that comes from an amyloid precursor protein. In healthy brains, these Aβ protein fragments are broken down and eliminated. However, in AD, the Aβ protein fragments aggregate to form hard, insoluble plaques. Aggregated forms of Aβ as well as soluble precursor forms called oligomeric Aβ act as microglial activators. The activated microglia have a beneficial effect of phagocytizing Aβ deposits, but they also have deleterious neuron-damaging effects, such as direct microglial neuron killing and by causing production of neurotoxic nitric oxide (NO) and inflammatory cytokines.

Microglia also play a roll in causing brain damage following hypoxic or anoxic insults to the brain. Hypoxic or anoxic brain insults may occur due to various causes, including but not limited to ischemic or hemorrhagic strokes, cardiac arrest and resuscitation, carbon monoxide poisoning, trauma, asphyxiation, strangulation, drowning, hemorrhagic shock, inhalant substance abuse (“huffing”), brain edema, iatrogenic disruption of cerebral circulation during surgery or other medical procedures like irradiation, etc.

Inhibition of certain cellular potassium channels has been proposed as an approach for reducing microglia-mediated neurotoxicity. Potassium channels are encoded by a super-family of 78 genes and are involved in diverse physiological processes ranging from repolarization following neuronal or cardiac action potentials, over regulating calcium signaling and cell volume, to driving cellular proliferation and migration. The voltage-gated Kv1.3 channel, is expressed in T and B lymphocytes, macrophages and microglia. However, in contrast to stronger immunosuppressants like calcineurin inhibitors and anti-TNF reagents, Kv1.3 inhibitors do not affect the ability of rodents or primates to respond to or to clear bacterial or viral infections and Kv1.3 is therefore regarded as a relatively safe drug target. Recent findings on the role of Kv1.3 in microglia activation in various experimental models have prompted us to study Kv1.3 in microglia as a potential target for AD.

AD is the most common cause of dementia among people aged 65 and older in all ethnic groups and is one of the most disabling and burdensome health conditions worldwide. AD is currently estimated to affect 4.5 million Americans and its incidence has more than doubled since 1980. Based on the increasing incidence of AD there is an urgent need for new therapeutics that can either prevent AD or slow its progression. All currently FDA-approved drugs for AD, the three acetylcholinesterase inhibitors Aricept, Razadyne, and Exelon, and the N-methyl-D-aspartate receptor antagonist, Namenda, only treat the symptoms of AD and cannot hold its progression.

It is desirable for therapies aimed at microglia-mediated neurotoxicity to meet the following goals:
(a) reduce the neurotoxic effects of microglia while at the same time maintaining their neuroprotective functions such as phagocytosis of amyloid-beta deposits;
(b) be specific to microglia so that its inhibition does not adversely affect important neuronal or astroglia functions; and
(c) not be broadly immunosuppressive.
   In this patent application, Applicants describe compositions and methods for reducing microglia-mediated neurotoxicity in a manner that meets some or all of these goals.

### SUMMARY OF THE INVENTIONS

In accordance with the present invention, there is provided a method for deterring microglia-mediated neurotoxicity in a human or non-human animal subject, said method comprising the step of inhibiting or blocking the intermediate-conductance calcium-activated potassium channel Kv1.3 in microglia. The inhibition or blocking of the Kv1.3 channels may be accomplished by administering to the subject a therapeutically effective amount of a Kv1.3 inhibiting substance. Examples of Kv1.3 inhibiting substances are described in U.S. Pat. No. 7,557,138 (Wulff et al.) and U.S. Pat. No. 8,067,460 (Wulff et al.) and in co-pending U.S. patent application Ser. No. 12/939,912, the entire disclosures of which are expressly incorporated herein by reference. Kv1.3 inhibition can cause relatively mild immunosuppression. Thus, the present invention is particularly suited to treatment of diseases, such as AD, that are characterized by Aβ-induced microglial neurotoxicity while not substantially deterring Aβ phagocytosis. Also, as described herein, in addition to symptomatic treatment, the methods of the present invention are effective to slow the onset or progression of those diseases.

Further in accordance with the present invention, the methods of the present invention may in some embodiments comprise administering to the subject, in an amount that is therapeutically effective to cause microglial Kv1.3 inhibition, a 5-phenoxyalkoxypsoralen compound of the following General Formula 1:

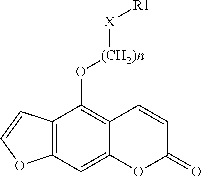

wherein:
n is 1 through 10, cyclic or acyclic and optionally substituted or unsubstituted;
X is O, S, N or C; and
R1 is aryl, heterocyclyl or cycloalkyl and is optionally substituted with one or more substituents selected from alkyl, alkoxy, amino and its alkyl derivatives, acylamino, carboxyl and its alkyl ester, cyano, halo, hydroxy, nitro and sulfonamido groups.
   Numerous specific compounds of General Formula 1 are described in the above-incorporated U.S. Pat. No. 7,557,138 (Wulff et al.) and U.S. Pat. No. 8,067,460 (Wulff et al.). Included among these compounds is 5-(4-Phenoxybutoxy)psoralen (PAP-1) (also sometimes referred to as 4-(4-Phenoxybutoxy)-7H-furo[3,2-g][1]benzopyran-7-on), which has the following structure:

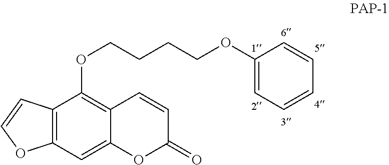 [SMILES: O=c1ccc2c(OCCCCOc3ccccc3)c3ccoc3cc2o1]

PAP-1 is a highly potent and selective small molecule Kv1.3 blocker. PAP-1 inhibits the Kv1.3 channel with an IC₅₀ of 2 nM and exhibits excellent selectivity over other ion channels, receptors and transporters. PAP-1 has a half-life of 3 hours in rats and of 6.7 hours in rhesus macaques. PAP-1 is orally bioavailable and has not exhibited long-term toxicity in rodents or primates. As described in greater detail below, PAP-1 reduces Aβ-induced microglia activation and subsequent neurotoxicity in both dissociated and organotypic hippocampal slice cultures, but does not block the ability of microglia to phagocytose Aβ. In pharmacokinetic studies PAP-1 has been shown to cross the blood brain barrier and to reach brain concentrations that equal or slightly exceed plasma-concentrations.

Still further in accordance with the present invention, the methods of the present invention may in some embodiments comprise administering to the subject, in an amount that is therapeutically effective to cause microglial Kv1.3 inhibition, a 4-phenoxybutoxy-substituted heterocyclic compound having the following General Formula 1:

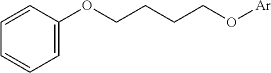

wherein Ar is selected from the group consisting of: phenyl, napthlalene-1-yl; anthraquinone-1-yl; phenanthrene-9-yl; quinoline-4-yl; isoquinolin-5-yl; quinazolin-4-yl; 1,2-dihydro-N-methyl-quinolin-2-one-4-yl; 2H-[1]benzopyran-2-one-4-yl; 2-phenyl-4H-[1]benzopyran-4-one-3yl; 2H-[1]benzopyran-2-one-5-yl; benzofuran-4-yl; furo[2,3-b]quinolin-4(9H)-one-9-yl; 7,8-dimethoxy-furo[2,3-b]quinoline-4-yl; furo[2,3-b]quinoline-4-yl; psoralen-8-yl; 5, 8-dimethoxy-psoralen-4-yl; 5-methoxy-4-methyl-psoralen-8-yl; 9H-xanthene-9-yl; 7-methyl-5H-furo[3,2-g][1]benzopyra-5-one-4-yl; 9-methoxy-7-methyl-5H-furo[3,2-g][1]benzopyran-5-one-4-yl; 5H-furo[3,2-g][1]benzopyran-5-one-4-yl; 2-methyl-6,7-methylendioxy-4H-[1]benzopyran-4-one-5-yl; 2,6-dihydro-8-methyl-pyrano[3,2-g][1]benzopyran-2,6-dione-5-yl and 7H-furo[3,2-g]chromene-7-thione-4-yl.

Further in accordance with the present invention, the methods of the present invention are in some embodiments carried out by administering a compound of General Formula 1 or of General Formula 2 or any pharmaceutically acceptable salt thereof alone or in combination with one or more pharmaceutically acceptable carriers, excipients and other ingredients commonly used in pharmaceutical preparations for oral, rectal, intravenous, intraarterial, intradermal, subcutaneous, intramuscular, intrathecal, sublingual, bucal, intranasal, trans-mucosal, trans-dermal, topical, other enteral, other parenteral and/or other possible route(s) of administration.

Further in accordance with the invention, in some embodiments, the inhibition or blockade of voltage-gated potassium channel Kv1.3 may be carried out in a manner that reduces neurotoxic effects of the microglia without preventing beneficial (e.g., phagocytic) effects of the microglia.

Still further in accordance with the invention, the method may be carried out to deter or slow neuron damage in subjects who suffer from a neurodegenerative disease. Some such subjects may have A[3 deposits (such as those suffering from Alzheimer's Disease or who are in the process of developing Alzheimer's Disease) and the inhibition or blockade of the voltage-gated potassium channel Kv1.3 may be carried out in a manner that reduces at least one neurotoxic effect of microglia (e.g., microglia-mediated neuronal killing, microglial production of NO and/or microglial cytokine production) while not preventing microglia from phagocytosing Aβ deposits.

Still further in accordance with the invention, in some embodiments, the method will be carried out to reduce neural damage in subjects who have suffered or are suffering an ischemic, anoxic or hypoxic conditions, events or insults, such as those who suffer a) ischemic stroke, b) hemorrhagic stroke, c) cardiac arrest and resuscitation, d) carbon monoxide poisoning, e) trauma, f) asphyxiation, g) strangulation, h) drowning, i) hemorrhagic shock, j) inhalant substance abuse or huffing, k) brain edema and 1) iatrogenic disruption of cerebral circulation during a surgery or other medical procedure.

Still further aspects and details of the present invention will be understood upon reading of the detailed description and examples set forth herebelow.

### BRIEF DESCRIPTION OF THE DRAWINGS

FIG. 1A is a bar graph showing NF-kB activation in Microglia after 2 hours of treatment with either control (vehicle only), AβO, AβO+doxycycline or AβO+PAP-1.

FIG. 1B is a bar graph comparing NO production by microglia after 24 hr of treatment with either control (vehicle only), AβO, AβO+doxycycline or AβO+PAP-1.

FIG. 2 is a bar graph comparing microglial cell-associated fluorescence as measured by flow cytometry following 2 hours of pre-treatment with either control (vehicle only), anti-SRA (scavenger receptor A) antibody, doxycycline or PAP-1.

FIG. 3 is a graph comparing the patch clamp response of the cultured microglia to a 500 millisecond pulse following treatment with control (saline only) and 1 μM PAP-1.

FIG. 4A is a graph showing that AbO and lipopolysaccharides (LPS) significantly increase Kv peak current density (**p<0.01) in microglia in a patch clamp assay.

FIG. 4B is a graph showing the patch clamp response of the cultured microglia to a 500 millisecond pulse following a 48 hour pre-treatment with control (saline).

FIG. 4C is a graph showing the patch clamp response of the cultured microglia to a 500 millisecond pulse following a 48 hour pre-treatment with AβO.

FIG. 4D is a graph showing the patch clamp response of the cultured microglia to a 500 millisecond pulse following a 48 hour pre-treatment with LPS.

FIG. 4E is a bar graph of qRT-PCR data showing that 24-hr treatment of microglia with AbO and LPS significantly increased the transcript level of Kv1.3 (n=3,p<0.05).

FIG. 5A shows Kv currents from an LPS activated microglial cell in response to voltage-steps from −80 mV to +100 mV in increments of 20 mV.

FIG. 5B shows a Boltzman plot of the data in FIG. 5A.

FIG. 5C shows the characteristic use-dependence of Kv1.3 in an LPS activated microglial cell. Currents were elicited every second by stepping the membrane from −80 mV to +40 mV.

FIG. 5D shows the effect of 100 pM of the Kv1.3 blocking peptide ShK-186 on the microglial Kv current.

FIG. 5E shows the effect of 1 nM of the Kv1.3 blocking peptide MgTX on the microglial Kv current.

FIG. 5F shows the effect of 10 nM of PAP-1 on the microglial Kv current.

FIG. 6 shows that freshly isolated microglia from the brains of 4 and 6 months old transgenic mice, which are widely used as a model for Alzheimer's disease express a higher Kv1.3 current density than microglia from 4 month-old control (wild-type) mice.

FIG. 7 shows that oligomeric AβO suppresses LTP (long-term synaptic potentiation) in rat brain slides and that treatment with PAP-1 prevents this suppression of LTP.

### DETAILED DESCRIPTION AND EXAMPLES

The following detailed description and the accompanying drawings to which it refers are intended to describe some, but not necessarily all, examples or embodiments of the invention. The described embodiments are to be considered in all respects only as illustrative and not restrictive. The contents of this detailed description and the accompanying drawings do not limit the scope of the invention in any way.

### General Methodology Used for Immunohistochemical Analysis (IHC) of Microglia Activation and Kv1.3 Expression

In the following examples and accompanying drawings, reference is made to various experiments wherein IHC analysis was carried out. In such examples, routinely cryopreserved mouse brain tissue was cryosectioned alternating for IHC and Western blot for evaluating microglia activation. Antibodies were used to 1) IBA-1 to reveal all microglia, 2) CD11b and SRA1 to reveal activated microglia, 3) TNF-α and IL-6 to reveal the classical activation state, 4) IL-4 and IL-13 for the alternative activation state, and 5) Kv1.3. Numbers of microglia in specific brain regions will be quantified using unbiased stereology in sections chosen from specified coordinates. We have successfully used two antibodies, a polyclonal antibody from Sigma and a monoclonal antibody, which was developed by the University of California at Davis/National Institutes of Health NeuroMab Facility (www.Neuromab.com) to detect Kv1.3. Multiplex immunostaining of Kv1.3 and microglia activation markers such as CD11b or SRA reveal the increased expression of Kv1.3 in activated microglia. Applicants also used unbiased and sensitive laser scanning cytometry (LSC) to analyze multiplex staining. In this automated method, location- and marker-specific quantitative data regarding immunoreactivity, size, and morphological features can be easily linked and compared.

Where morphometric analysis of dendrites was carried out, Paraffin embedded hippocampus was Golgi-stained and analyzed by Neurolucida utilizing the Sholl method of concentric circles.

In instances where electrophysiology is used, CD11b^{|} microglia were isolated using Percoll separation and anti-CD11b magnetic beads, attached to polylysine coated cover slips and immediately used for whole-cell patch-clamp experiments. This method effectively eliminates contaminating astrocytes and endothelial cells and takes less than 4 hrs. A small aliquot will be fixed for later flow cytometrical analysis for purity of microglia. Kv1.3 channels will be recorded in the whole-cell mode of the patch-clamp technique. The molecular identity of the currents will be further confirmed by their sensitivity to the Kv1.3 blockers used in the following examples. With this approach we envision that we will be able to study at least 40-50 cells per preparation. In parallel to the electrophysiological experiments we will also determine the expression of Kv1.3 and other Kv-1 family channels like Kv1.5 by qRT-PCR as previously described (48).

### Kv1.3 Expression is Increased in Plaque-Associated Microglia

Applicants investigated 5XFAD and APPswe/PS1De9 mice, which are animal models of AD. Hippocampal sections from 5xFAD mice and wild-type (Wt) littermates were stained with anti-Kv1.3 and the amyloid dye FSB. FSB demonstrated the typically small amyloid plaques in 5xFAD mice. In 5xFAD mice, the Kv1.3 stain was coarsely granular in contrast to the finely diffuse stain seen in wt mice. Also, microglia surrounding an amyloid plaque in hippocampal sections from APPswe/PS1De9 mice were doubly stained with anti-Kv1.3 and CD11b. Kv1.3 was localized to CD11b-immunoreactive activated microglia closely associated with amyloid plaques.

Separate experiments showed that Kv1.3 antibodies did not stain neurons, astrocytes, or oligodendrocytes, in keeping with its reported expression pattern in the brain.

### Kv1.3 Blockade Inhibits Aβ-Induced Microglia Activation and Microglia-Mediated Neurotoxicity

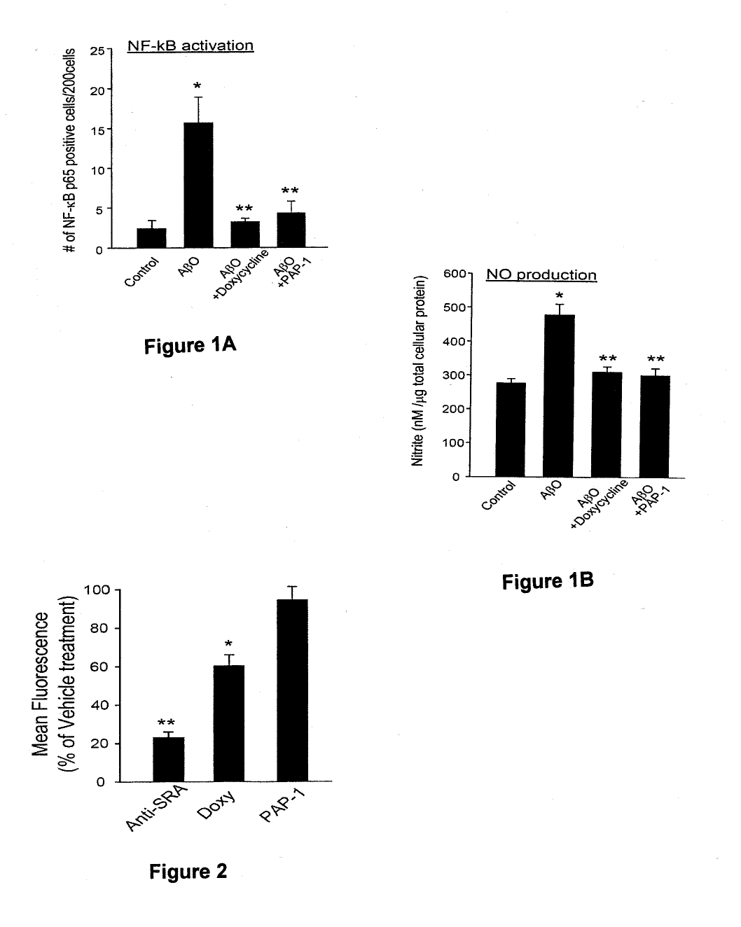

Applicants found that the specific Kv1.3 blocker PAP-1 inhibited signs of microglia activation induced by AβO in cultured mouse microglia, such as proliferation and morphological transformation, as well as NFκ-B activation and nitric oxide (NO) production. In addition, PAP-1 also blocked increased microglia release of tumor necrosis factor-α, NFκ-B activation, and NO production induced by fAβ stimulation. FIGS. 1A and 1B show data regarding AβO-induced NFκ-B and NO, respectively.

FIG. 1A is a bar graph showing NF-kB activation in Microglia after 2 hours of treatment with either control (vehicle only), AβO, AβO+doxycycline or AβO+PAP-1. Two (2) hours after administration of the indicated treatment, the mouse brains were sectioned and immunostained with an antibody for p65 of NFκB to mark cells with NFκB activation. Numbers of p65-positive cells per 200 DAPI-labeled cells were determined.

FIG. 1B is a bar graph comparing NO production by microglia after 24 hr of treatment with either control (vehicle only), AβO, AβO+doxycycline or AβO+PAP-1. Measurements were made in the conditioned medium and normalized to the amount of total cellular protein in each culture. n=4-6, *p<0.001 compared with control, **p<0.001 compared with the “AβO” group. AβO, 20 nM, doxycycline, 20 μM, and PAP-1, 1 μM. NO released by AβO-treated microglia is the major soluble mediator of AβO-induced microglial neurotoxicity. This toxicity was blocked by co-treating AβO-stimulated cultured microglia with PAP-1.

Applicants also performed in situ experiments using hippocampal slices, which better reflect the conditions in the brain in terms of microglial density and their interaction with astroglia and neurons, showed that PAP-1 treatment substantially reduced AβO-induced microglia activation and blocked AβO-induced neuronal damage (indicated by propidium iodide uptake and Fluoro-Jade C staining). Three consecutive hippocampal slices were obtained from mouse brain and from mice received the same indicated treatment. One slice was used for CD11b staining (green) for activated microglia (slices outlined by Hoechst stain), one for propidium iodide (PI) uptake, and one for Fluoro-Jade C stain for neuronal damage. DG: dentate gyrus. AβO, 20 nM; doxycycline, 20 μM; and PAP-1, 1 μM. Because of the restricted microglial expression of Kv1.3 in the brain, these observations support a conclusion that the PAP-1 effect was through inhibiting microglial Kv1.3.

### PAP-1 Did Not Impair the Ability of Microglia to Phagocytose Aβ

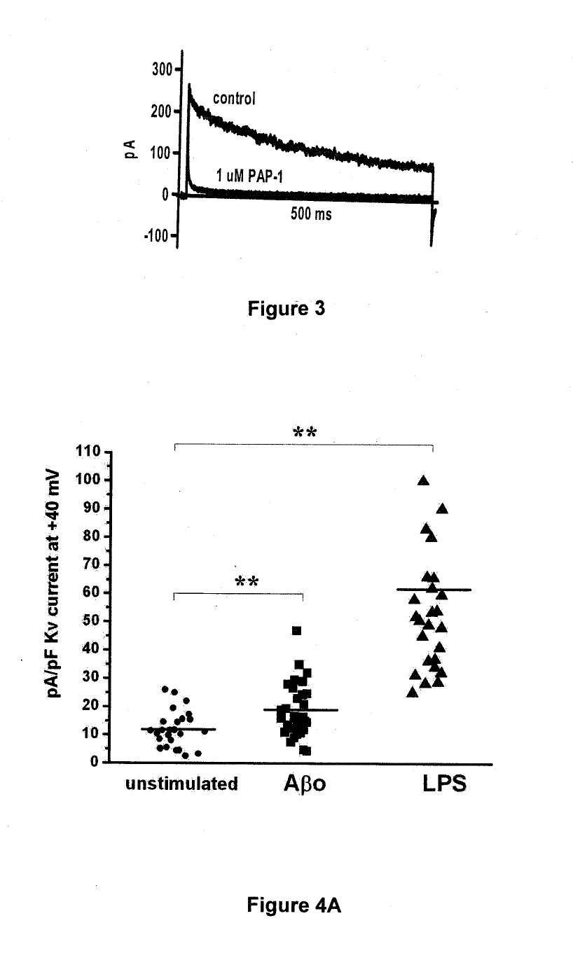

Using an Aβ uptake assay, Applicants pretreated microglia with either control (vehicle only), anti-SRA (scavenger receptor A) antibody, doxycycline or PAP-1. FIG. 2 is a bar graph showing cell-associated fluorescence as measured by flow cytometry after 2 hrs of such pre-treatment. (n=3, *p<0.01 and **p<0.001 compared with control). The anti-SRA antibody and doxycycline pretreatments caused significant decreases in Aβcell-associated fluorescence but PAP-1 pretreatment did not. Thus, PAP-1 did not impair the ability of microglia to phagocytose Aβ but doxyxyxline did. These data suggest that using PAP-1 for treatment of amyloid neurodegenerative diseases (such as AD) may have an advantage over doxycycline treatment in that PAP-1 does not hamper Aβ clearance by microglia.

### Kv1.3 is Expressed and Functional in Mouse Microglia

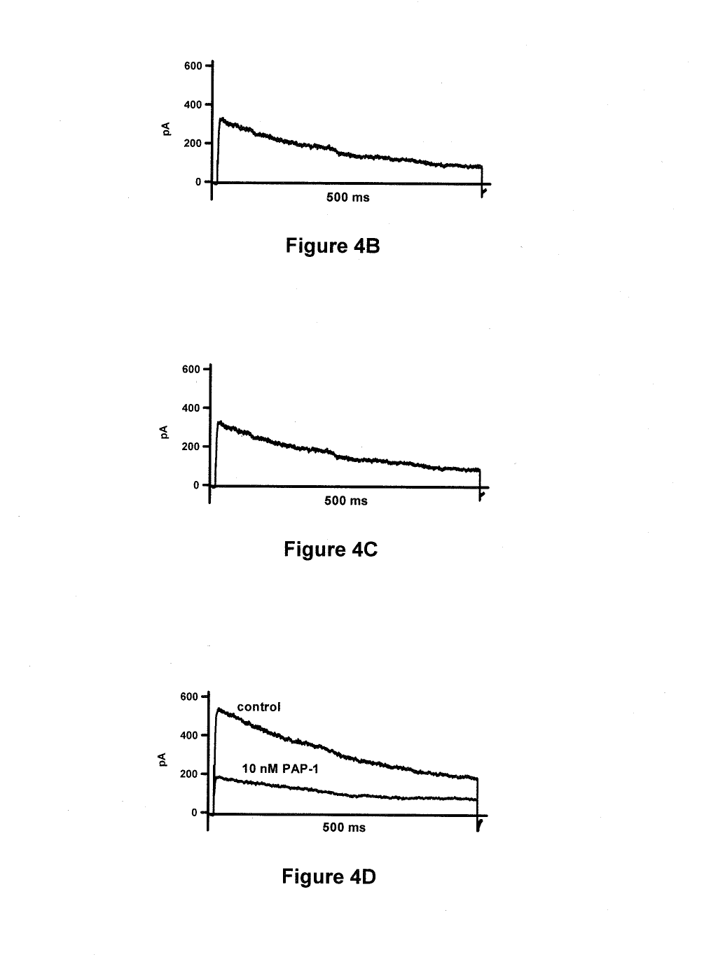

In order to determine if Kv1.3 is indeed functionally expressed in mouse microglia Applicants performed electrophysiological experiments on cultured mouse microglia in the whole-cell mode of the patch-clamp technique. Kv currents were elicited with 500 millisecond pulses from a holding-potential of −100 mV to +40 mV applied every 45 sec. Under these conditions a Kv current exhibiting the characteristic use-dependence and inactivation of Kv1.3 was observed in a majority of cells. FIG. 3 is a graph comparing the response of the cultured microglia to a 500 millisecond pulse following treatment with control (saline only) and 1 μM PAP-1. These data demonstrate that Kv1.3 is expressed in mouse microglia.

Stimulation with AbO and LPS Increases Kv1.3 Expression in Cultured Microglia

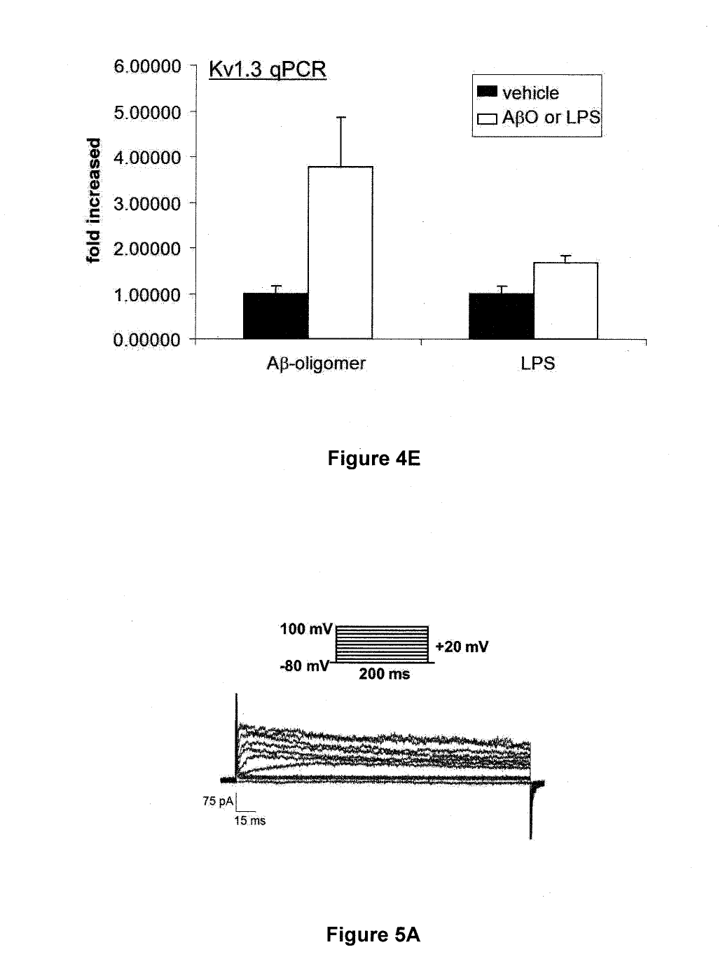

Microglia cultured from newborn C57B1/6 mice were treated with lipopolysaccharides (LPS) or AbO for 24 or 48 hrs. FIG. 4A is a graph showing that AbO and LPS significantly increase Kv peak current density (**p<0.01) determined by whole-cell voltage-clamp recordings. Kv currents were elicited by 500 ms voltage steps from −80 to 40 mV (representative traces on right).

FIG. 4B shows the patch clamp response of the cultured microglia to a 500 millisecond pulse following a 48 hour pre-treatment with control (saline).

FIG. 4C shows the patch clamp response of the cultured microglia to a 500 millisecond pulse following a 48 hour pre-treatment with AβO.

FIG. 4D shows the patch clamp response of the cultured microglia to a 500 millisecond pulse following a 48 hour pre-treatment with LPS demonstrating that microglial activation increases Kv1.3 expression.

Additionally, cultured microglia were immuno-fluorescently stained for Kv1.3 and the microglial marker CD11b, and counterstained with DAPI in accordance with the techniques described generally above. Both LPS and AbO stimulated the activated morphology of microglia and enhanced Kv1.3 immunoreactivity.

FIG. 4E shows qRT-PCR data indicating that 24-hr treatment of microglia with AbO and LPS significantly increased the transcript level of Kv1.3 (n=3,p<0.05).

### The Kv Channel in AbO-Stimulated Microglia Exhibits the Biophysical Properties of Kv1.3

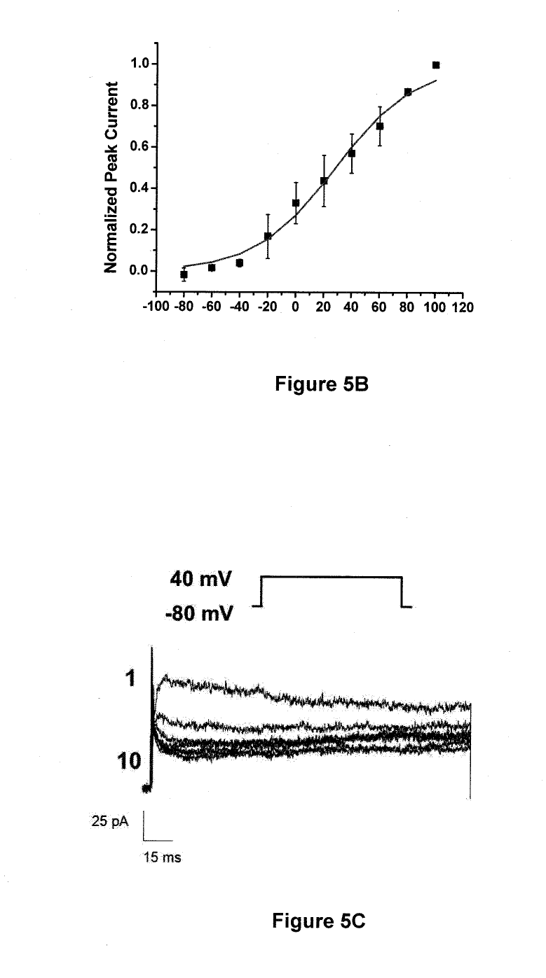

FIGS. 5A through 5C show Applicants conducted biophysical characterization of cultured microglia by whole-cell voltage-clamp recordings of currents elicited by voltage steps from −80 to 100 mV in 20 mV increments with Boltzmann fit of normalized peak currents: AbO (V1/2-25.6 mV) Use-dependent inactivation elicited by repetitive depolarization from −80 to +40 mV (1 pulse/sec) for 10 pulses. FIGS. 5A through 5F show a pharmacological characterization of currents from cultured microglia stimulated for 48 hours with AbO or LPS (IC₅₀s for the two conditions): ShK-186 (68.5 pM and 79.2 pM), MgTX (79.7 pM and 78.9 pM), PAP-1 (6.8 nM and 9.5 nM), respectively. ShK-186 is a novel analog of Shk, a natural peptide isolated from the sea anemone, Stichodactyla heliantus. ShK-186 has been shown to be a selective and potent blocker of the Kv1.3 potassium channel.

Microglia in 5xFAD Mice Express More Functional Kv1.3 than WT Microglia

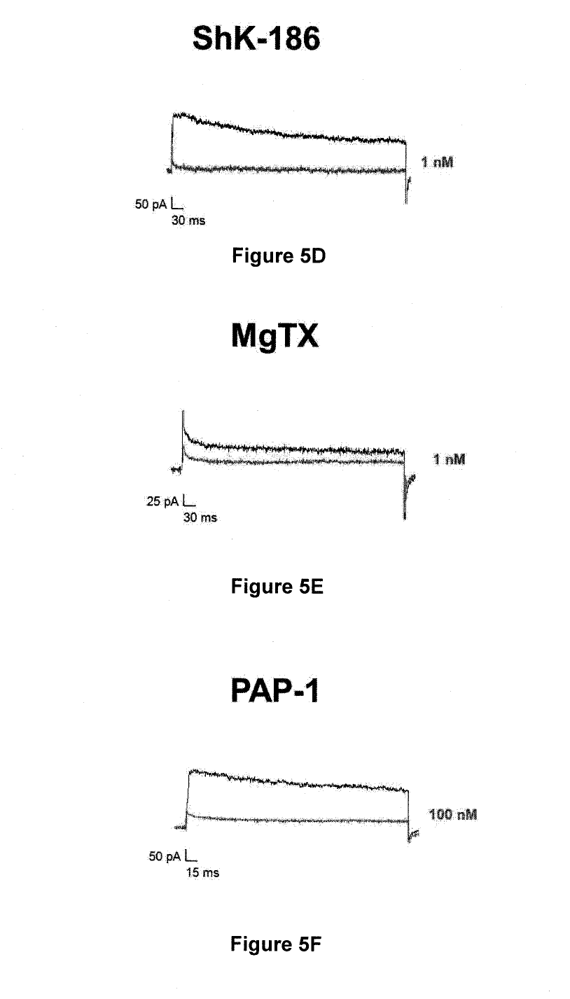

FIG. 6 shows Peak K+ current densities from microglia isolated from WT and 5xFAD mice, determined by whole-cell voltage-clamp recordings, elicited by 500 ms voltage step −80 to 40 mV.

Additionally, cerebral sections from 4 month-old WT and 5xFAD mice were immunostained with anti-Kv1.3 (red) and the amyloid dye FSB (blue) and examined in accordance with the general methods described above. Representative images of an amyloid plaque were also costained for Kv1.3 (red) and CD11b (green) and counterstained with DAPI (blue). Enhanced Kv1.3 immunoreactivity was observed in microglia around the FSB-positive amyloid plaques.

These data indicate that 5xFAD Mice Express More Functional Kv1.3 Than WT Microglia.

PAP-1 Prevents the Inhibitory Action of AID on the Induction of CA1 LTP in Rat Hippocampal Slices

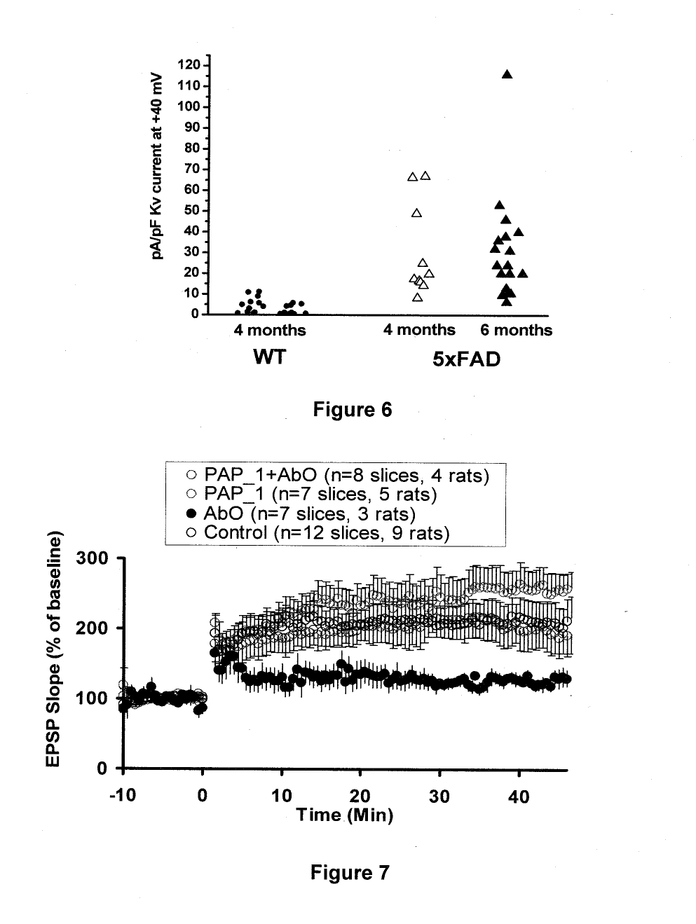

FIG. 7 summarizes an experiment wherein, under control conditions, CA1 LTP was induced by high frequency stimulation (HFS), which consists of 4 trans of 100Hz basal intensity stimulation lasting for 1 s per train. In control (vehicle only) group, following the HFS, the amplitude or slope of fEPSP was increased to 209±28% of baseline at 45 min after HFS. The bath application of AbO (50 nM) for 10 min blocked the induction of LTP (126±6.7% of control). Pretreatment with PAP-1 (10 μM) for 30 min prior to AbO application prevented the inhibition of AbO on the induction of LTP. The mean of five consecutive measurements at the end of LTP induction (45 min) was normalized to the baseline (100%) which was the mean of five consecutive measurements just before the HFS.

It is to be appreciated that, although the invention has been described hereabove with reference to certain examples or embodiments of the invention, various additions, deletions, alterations and modifications may be made to those described examples and embodiments without departing from the intended spirit and scope of the invention. For example, any elements, steps, members, components, compositions, reactants, parts or portions of one embodiment or example may be incorporated into or used with another embodiment or example, unless otherwise specified or unless doing so would render that embodiment or example unsuitable for its intended use. Also, where the steps of a method or process have been described or listed in a particular order, the order of such steps may be changed unless otherwise specified or unless doing so would render the method or process unsuitable for its intended purpose. Additionally, the elements, steps, members, components, compositions, reactants, parts or portions of any invention or example described herein may optionally exist or be utilized in the substantial absence of other elements, steps, members, components, compositions, reactants, parts or portions unless otherwise noted. All reasonable additions, deletions, modifications and alterations are to be considered equivalents of the described examples and embodiments and are to be included within the scope of the following claims.

What is claimed is:

1. A method for deterring microglia-mediated neurotoxicity in a human or non-human animal subject, said method comprising the step of inhibiting or blocking the voltage-gated potassium channel Kv1.3 in microglia.

2. A method according to claim 1 wherein the step of inhibiting or blocking the voltage-gated potassium channel Kv1.3 comprises administering to the subject a therapeutically effective amount of a substance that inhibits or blocks the Kv1.3 channel.

3. A method according to claim 2 wherein the substance comprises a 5-phenoxyalkoxypsoralen compound having the formula: 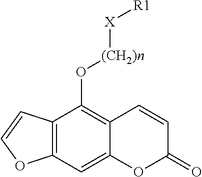
wherein:
n is 1 through 10, cyclic or acyclic and optionally substituted or unsubstituted;
X is O, S, N or C; and
R1 is aryl, heterocyclyl or cycloalkyl and is optionally substituted with one or more substituents selected from alkyl, alkoxy, amino and its alkyl derivatives, acylamino, carboxyl and its alkyl ester, cyano, halo, hydroxy, nitro and sulfonamido groups.

4. A method according to claim 3 wherein the compound comprises (4-Phenoxybutoxy)psoralen (PAP-1)

5. A method according to claim 2 wherein the substance comprises a 1 4-phenoxybutoxy-substituted heterocyclic compound having the formula: 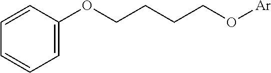
wherein Ar is selected from the group consisting of: phenyl, napthlalene-1-yl; anthraquinone-1-yl; phenanthrene-9-yl; quinoline-4-yl; isoquinolin-5-yl; quinazolin-4-yl; 1,2-dihydro-N-methyl-quinolin-2-one-4-yl; 2H-[1]benzopyran-2-one-4-yl; 2-phenyl-4H-[1]benzopyran-4-one-3yl; 2H-[1]benzopyran-2-one-5-yl; benzofuran-4-yl; furo[2,3-b]quinolin-4(9H)-one-9-yl; 7,8-dimethoxy-furo[2,3-b]quinoline-4-yl; furo[2,3-b]quinoline-4-yl; psoralen-8-yl; 5, 8-dimethoxy-psoralen-4-yl; 5-methoxy-4-methyl-psoralen-8-yl; 9H-xanthene-9-yl; 7-methyl-5H-furo[3,2-g][1]benzopyra-5-one-4-yl; 9-methoxy-7-methyl-5H-furo[3,2-g][1]benzopyran-5-one-4-yl; 5H-furo[3,2-g][1]benzopyran-5-one-4-yl; 2-methyl-6,7-methylendioxy-4H-[1]benzopyran-4-one-5-yl; 2,6-dihydro-8-methyl-pyrano[3,2-g][1]benzopyran-2,6-dione-5-yl and 7H-furo[3,2-g]chromene-7-thione-4-yl.

6. A method according to claim 1 wherein the inhibition or blockade of the potassium channel Kv1.3 reduces neurotoxic effects of the microglia without preventing beneficial effects of the microglia.

7. A method according to claim 4 wherein the subject has Aβ deposits and wherein the inhibition or blockade of the potassium channel Kv1.3 reduces at least one neurotoxic effect of microglia selected from a) microglia-mediated neuronal killing, b) microglial production of NO and c) microglial cytokine production while not preventing microglia from phagocytosing Aβ deposits.

8. A method according to claim 1 wherein the method is performed to reduce neural damage in a subject suffering from a neurodegenerative disease.

9. A method according to claim 6 wherein the neurodegenerative disease is Alzheimer's Disease.

10. A method according to claim 1 wherein the method is performed to reduce neural damage in a subject who has suffered or is suffering an ischemic, anoxic or hypoxic insult.

11. A method according to claim 10 wherein the ischemic, anoxic or hypoxic insult is due to at least one cause selected from a) ischemic stroke, b) hemorrhagic stroke, c) cardiac arrest and resuscitation, d) carbon monoxide poisoning, e) trauma, f) asphyxiation, g) strangulation, h) drowning, i) hemorrhagic shock, j) inhalant substance abuse or huffing, k) brain edema and l) iatrogenic disruption of cerebral circulation during a surgery or other medical procedure.

12. The use of an agent that inhibits or blocks potassium channel Kv1.3 in microglia in the manufacture of a pharmaceutical preparation for treating a microglia-mediated neurotoxicity in a human or non-human animal subject.

13. A use according to claim 12 wherein the agent comprises a 5-phenoxyalkoxypsoralen compound having the formula: 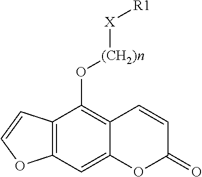
wherein:
n is 1 through 10, cyclic or acyclic and optionally substituted or unsubstituted;
X is O, S, N or C; and
R1 is aryl, heterocyclyl or cycloalkyl and is optionally substituted with one or more substituents selected from alkyl, alkoxy, amino and its alkyl derivatives, acylamino, carboxyl and its alkyl ester, cyano, halo, hydroxy, nitro and sulfonamido groups.

14. A use according to claim 12 wherein the agent comprises (4-Phenoxybutoxy)psoralen (PAP-1).

15. A use according to claim 12 wherein the agent comprises a 1 4-phenoxybutoxy-substituted heterocyclic compound having the formula: 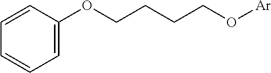
wherein Ar is selected from the group consisting of: phenyl, napthlalene-1-yl; anthraquinone-1-yl; phenanthrene-9-yl; quinoline-4-yl; isoquinolin-5-yl; quinazolin-4-yl; 1,2-dihydro-N-methyl-quinolin-2-one-4-yl; 2H-[1]benzopyran-2-one-4-yl; 2-phenyl-4H-[1]benzopyran-4-one-3yl; 2H-[1]benzopyran-2-one-5-yl; benzofuran-4-yl; furo[2,3-b]quinolin-4(9H)-one-9-yl; 7,8-dimethoxy-furo[2,3-b]quinoline-4-yl; furo[2,3-b]quinoline-4-yl; psoralen-8-yl; 5, 8-dimethoxy-psoralen-4-yl; 5-methoxy-4-methyl-psoralen-8-yl; 9H-xanthene-9-yl; 7-methyl-5H-furo[3,2-g][1]benzopyra-5-one-4-yl; 9-methoxy-7-methyl-5H-furo[3,2-g][1]benzopyran-5-one-4-yl; 5H-furo[3,2-g][1]benzopyran-5-one-4-yl; 2-methyl-6,7-methylendioxy-4H-[1]benzopyran-4-one-5-yl; 2,6-dihydro-8-methyl-pyrano[3,2-g][1]benzopyran-2,6-dione-5-yl and 7H-furo[3,2-g]chromene-7-thione-4-yl.

16. A use according to claim 12 wherein the agent is to be administered at a dose that reduces neurotoxic effects of the microglia without preventing beneficial effects of the microglia.

17. A use according to claim 16 wherein the preparation is for treatment of a microglia-mediated neurotoxicity characterized by the formation of Aβ deposits and wherein the agent is to be administered at a dose that lessens at least one neurotoxic effect of microglia selected from a) microglia-mediated neuronal killing, b) microglial production of NO and c) microglial cytokine production while not preventing microglia from phagocytosing Aβ deposits.

18. A use according to claim 12 wherein the pharmaceutical preparation is for reducing neural damage in subjects suffering from a neurodegenerative disease.

19. A use according to claim 12 wherein the pharmaceutical preparation is for reducing neural damage in subjects suffering from Alzheimer's Disease.

20. A use according to claim 12 wherein the pharmaceutical preparation is for reducing neural damage in subjects who have suffered an ischemic, anoxic or hypoxic insult.

21. A use according to claim 12 wherein the pharmaceutical preparation is for reducing neural damage in subjects who have suffered an ischemic, anoxic or hypoxic insult as a result of a) ischemic stroke, b) hemorrhagic stroke, c) cardiac arrest and resuscitation, d) carbon monoxide poisoning, e) trauma, f) asphyxiation, g) strangulation, h) drowning, i) hemorrhagic shock, j) inhalant substance abuse or huffing, k) brain edema or l) iatrogenic disruption of cerebral circulation during a surgery or other medical procedure.
